(19) United States
(12) Patent Application Publication
(10) Pub. No.: US 2018/0207361 A1
(43) Pub. Date: Jul. 26, 2018

(54) ACTUATING DEVICE FOR ADMINISTERING A BOLUS

(71) Applicant: B. Braun Melsungen AG, Melsungen (DE)

(72) Inventors: Karsten Haslbeck, Melsungen (DE); Martin Sippel, Melsungen (DE)

(21) Appl. No.: 15/746,213

(22) PCT Filed: Jul. 19, 2016
(86) PCT No.: PCT/EP2016/067195

§ 371 Date: Jan. 19, 2018

(30) Foreign Application Priority Data
Jul. 21, 2015 (DE) 10 2015 213 726.0

(51) Int. Cl. A61M 5/168; A61M 5/142; A61M 5/148

(52) U.S. Cl. CPC A61M 5/16881; A61M 2005/1405; A61M 5/148; A61M 5/14216

(57) ABSTRACT

A device for administering a bolus, includes an actuating element to actuate a closing mechanism which opens and closes a hose segment. The hose segment can be connected to a fluid source and to a patient. The device also includes a movable element, a blocking element which locks the movable element in an upper position and which can be released by the actuating element, an elastic element which pushes the actuating element and movable element apart, a first actuation blocking element which locks the actuating element in a lower position and can be released by the movable element, and a bolus reservoir for holding a bolus quantity of fluid. The volume of the bolus reservoir can be reduced by displacing the movable element. A second actuation blocking element locks the actuating element in the upper position can be released by the movable element.

### RELATED APPLICATIONS

This application is the United States national phase entry of International Application No. PCT/EP2016/1067195, filed Jul. 19, 2016, which is related to and claims the benefit of priority of German Application No. 10 2015 213 726.0, filed Jul. 21, 2015. The contents of International Application No. PCT/EP2016/067195 and German Application No. 10 2015 213 726.0 are incorporated by reference herein in their entireties.

### FIELD

The invention relates to an actuating device for administering a fluid bolus.

### BACKGROUND

Medicaments in thud form are administered to a patient from a fluid reservoir with the aid of a hose system. In the treatment of pain (analgesia), for example, there is a need for a continuous, permanent supply of the fluid and for a supplementary administration of a fixed bolus quantity of the fluid. The aim of analgesia is to provide patients continuously with a painkiller in order to suppress pain. To alleviate episodes of pain that go beyond an average level of pain, it is known to provide the patient with an actuating element whose actuation triggers the administration of an additional dose of painkiller (bolus). For this purpose, the fluid reservoir is provided, in which the dose of fluid for the bolus is contained. The fluid reservoir can be connected to the fluid delivery pump via an inflow line and can be connected to a patient port via an outflow line. The patient port serves for connection to a patient or to a catheter inserted in the patient.

### SUMMARY

The object of the invention is to make available an actuating device which serves for triggering a bolus in patient-controlled analgesia (PCA) and with which the risk of an overdose is reduced.

A segment of a hose which conveys the fluid to be administered, and which can be connected to the fluid source via an inflow line and to a patient via an outflow line, can be closed by a closing mechanism. The closing mechanism is actuated by an actuating, element which is displaceable along a longitudinal axis from an upper (proximal) position to a lower (distal) position. The upper position of the actuating button can also be designated as the first position, while its lower position can be designated as the second position. The actuating element actuates the closing mechanism in such a way that the hose segment is opened in the lower position of the actuating element and is closed in the upper position thereof.

The upper position of the actuating element is a starting position. In the starting position of the actuating element, the closing mechanism closes the hose segment. No fluid then flows from the source through the hose segment in the direction of the patient. The fluid supply is interrupted. When the actuating element is actuated, it is displaced in the direction of its lower position, as a result of which the closing mechanism is actuated and frees the hose segment, such that fluid from the source flows through the hose segment in the direction of the end that is connectable to the patient. The actuating device according to the invention can also be designated as a trigger device for triggering a bolus. The actuating element can be an actuating button.

The movable element is displaceable along a longitudinal axis from an upper position to a lower position, independently of the actuating element. The longitudinal axis along which the movable element is displaceable is preferably parallel to the longitudinal axis along which the actuating element is displaceable. The two longitudinal axes can be identical, for example.

A blocking means is provided in order to lock the movable element in its upper position. The blocking means is released by displacement of the actuating element in the direction of the lower position thereof. An elastic element is arranged between the actuating element and the movable element in such a way that actuating element and movable element are pushed apart, that is to say away from each other.

A bolus reservoir contains a bolus quantity of the fluid, which is forced out of the bolus reservoir by displacement of the movable element in the direction of the lower position thereof. In doing so, the movable element reduces the volume of the bolus reservoir.

The actuating element is provided with two blocking means. A first actuation blocking, means locks the actuating element in the lower position thereof, that is to say after the actuating element has been pressed in. The first actuation blocking means is releasable by the movable element when the latter reaches its lower position. The second actuation blocking means locks the actuating element in the upper position thereof and is releasable from the movable element when the latter reaches its upper position. The actuation blocking means can be button locks for blocking the actuating button.

When the actuating element is pressed inward in the direction of its lower position, this actuates the closing mechanism. The clamped-off segment of the hose freed, and fluid flows from the direction of the source via the inflow line and the outflow line in the direction of the end that is connectable to the patient.

The movable element is initially locked in its upper position (starting position) by the blocking means. When the actuating element is pressed inward, the blocking means of the movable element is released and the elastic element presses the movable element away from the actuating element. When the button reaches its lower position, it is locked there by the first actuation blocking means of the actuating element. The first actuation blocking means then forms art abutment for the force of the elastic element, such that the movable element is advanced in the direction of the lower position thereof by the elastic element bearing on the locked actuating element. In doing so the movable element presses the bolus quantity of the fluid out of the bolus reservoir.

As soon as the movable element has reached the lower position thereof, this releases the first actuation blocking means of the actuating element, and the actuating element is pushed back in the direction of the upper position thereof by the elastic element. The movable element supports itself at the lower return point thereof. The lower return point of the movable element can be formed, for example, by a stop on the bolus reservoir.

After the bolus is administered completely, the actuating element is thus automatically pushed back to the upper position thereof and is locked there by the second actuation blocking means. In the upper position, the closing mechanism is no longer actuated, such that the hose segment is closed again and no more fluid flows in the direction of the end that is connectable to the patient.

As soon as new fluid flows into the bolus reservoir, the movable element is displaced, counter to the force of the elastic element, from the lower position thereof toward the upper position thereof. Triggering of a new bolus by actuation of the actuating element is prevented here because the second actuation blocking means locks the actuating element. It is only when the complete bolus quantity has flowed in and the movable element has again reached the upper position that the latter frees the second actuation blocking means of the actuating element and, by pressing in the actuating element, the administration of a further bolus can be triggered.

The bolus reservoir is here preferably connected to the inflow line and the outflow line via a bolus line. Fluid flowing through the inflow line from a source or fluid thus passes via the bolus into the bolus reservoir, whereas, upon administration of the bolus, the fluid flows through the outflow line in the direction of the end that is connectable to the patient. The flow resistance of the hose segment should be less than the flow resistance of the bolus line. Fluid flowing through the inflow line chooses the path of least flow resistance and consequently flows through the hose segment as long as the latter is freed by the closing mechanism. It is only when the closing mechanism clamps off the hose segment, after return of the actuating element to the upper position thereof, that the fluid flows through the bolus line with greater flow resistance into the bolus reservoir in order to fill the latter.

The closing mechanism for the hose segment can be provided with a clamping spring which clamps off the hose segment. The spring force of the clamping spring counteracts a displacement of the actuating element from the upper position thereof toward the lower position thereof. When the actuating element is pressed in, the spring force of the clamping spring therefore has to be overcome in order to free the hose segment. On account of the spring force of the clamping spring, the actuating element seeks to return to the starting position (upper position).

The clamping spring can be provided with a spring arm that clamps off the hose segment. The actuating element can have an actuating fork which engages with the spring arm and which, when the actuating element is pressed inward in the direction of the lower position, presses against the spring arm and actuates the clamping spring in order to open the closing mechanism and free the hose segment.

The blocking means can have a latch mechanism provided with a latching lug which is held on a spring clip and which holds the movable element. The latching lug prevents the movable element from being displaced by the force of the elastic element. A laterally protruding latching web of the movable element can thereby engage on the latching lug. When the actuating element is pressed in, the latching web of the movable element is pushed beyond the latching lug, because the spring clip yields. When the latching lug is overcome, the movable element is freed and the elastic element presses the movable element into the bolus reservoir in order to force fluid out of the bolus reservoir.

The movable element and the actuating element are each provided with an engagement surface. The engagement surfaces of the movable element and of the actuating element are designed to be placed against each other such that a force acting on the actuating element is transmitted to the movable element, and vice versa. When the actuating element is pressed inward in order to release the dosing mechanism of the hose segment, the actuation force is transmitted via the engagement surfaces to the movable element, such that the actuating element displaces the movable element in the direction of the lower position thereof. The blocking means of the movable element is released automatically in the process. The elastic clement then displaces the movable element farther in the direction of the lower position, wherein the elastic element bears on the actuating element. As soon as the actuating element reaches the lower position, that is to say has been pressed in completely, it is locked by the first actuation blocking means, and the movable element is able to bear on the locked actuating element while it displaces the movable element in order to administer the bolus.

The first actuation blocking means of the actuating element can be provided here with a resilient latching pawl which engages on a housing part. The latching pawl locks the actuating element with respect to the housing part and prevents the elastic element from displacing the actuating element back to the upper position thereof.

The movable element preferably has a first engagement surface which actuates the latching pawl. As soon as the movable element reaches the lower position thereof, it automatically actuates the latching pawl counter to the spring force thereof via the first engagement surface. The actuating element is thereby freed again, and the elastic element pushes the actuating element back to the upper position thereof.

The second actuation blocking means locks the actuating element in the upper position thereof. This can be effected by a spring clip which locks the actuating element in the upper position with respect to the housing. A second engagement surface arranged on the movable element can be designed to actuate the spring clip counter to the spring force thereof as soon as the movable element reaches the upper position thereof. Thus, when the fluid flowing into the bolus reservoir displaces the movable element in the direction of the upper position thereof, the movable element actuates the spring clip via the second engagement surface. The spring clip thereby released from the housing, and the actuating element is freed for a renewed actuation.

The movable element can be a piston or a balloon. In the case of a piston, an actuation of the actuating element leads to the displacement of the piston. In the case of a balloon, an actuation of the actuating element leads to the displacement or pressing-in or deformation of the balloon.

The movable element can be mounted in the fluid reservoir in such a way as to be displaceable counter to the force of an elastic element. In the case of the piston, the elastic element can be a spring that displaces the piston. In the case of the balloon, the elastic element can be a compressible part of the balloon itself.

At least a part of the piston is guided with a sliding movement in the bolus reservoir. The bolus reservoir then forms a cylinder for the piston. The bolus reservoir can in particular be designed completely as a cylinder in which the piston is displaceable to and fro.

In a particularly preferred embodiment, the actuating element can be actuated along a first actuation path and along a further, second actuation path that goes beyond the first actuation path. During travel along the first actuation path, the movable element is displaced manually, that is to say exclusively by manual actuation by the user applying a corresponding force by hand. In the process, a valve at the outlet side to the patient is already opened. By manual displacement of the movable element along the first actuation path, a first bolus quantity is thus administered manually, this first bolus quantity can be, for example, a residual fluid still present in the fluid line. It is only after the first actuation path has been traveled that the actuating element triggers an automatic administration of a second bolus quantity. The movable element is here displaced automatically along the second actuation path. In this context, automatically means that the movable element can be displaced by a spring, for example, and no force needs to be applied manually by the user for this purpose. During the travel along the second actuation path, the quantity of fluid contained in the fluid reservoir is thus administered automatically as a second bolus quantity.

It is particularly desirable here if, during travel along the first actuation path, a mechanical resistance has to be overcome in order to displace the actuating element. This mechanical resistance can be formed, for example, by resilient blocking elements which are to be displaced and overcome by the actuating element in the region of the first actuation path. This mechanical resistance provides haptic feedback, which indicates to the user the imminent administration of the main bolus quantity (second bolus quantity). The three for actuating the movable element is then increased for the first actuation path.

The movable element or the piston is preferably mounted so as to be displaceable counter to the force of a spring. The spring force of the spring serves, on the one hand, to counteract the pressure of the fluid flowing in, such that the movable element is displaced over a predefined period of time. On the other hand, the spring force serves to actuate the movable element when the actuating element is pressed, such that the fluid is pressed out of the fluid reservoir into the outflow line on account of the spring force. A first spring force can counteract the movable element during the actuation along the first actuation path, in order to provide a mechanical resistance for the manual actuation. A second spring force can support or automatically displace the movable element during the actuation of the actuating element along the second actuation path. An advantage of the manual bolus administration along the first actuation path in combination with the automatic bolus administration along the second actuation path is that resistances acting on the movable element, such as, in particular, the static friction of the at least one movable element, or injection resistances in the attached catheter can be better overcome.

Preferably, an inlet valve is provided in the inflow line and an outlet valve in the outflow line in order to interrupt and free the fluid path. The inlet valve and the outlet valve are each connected to the actuating element in such a way that, in the non-actuated state of the actuating element, the inlet valve frees the inflow line and the outlet valve blocks the outflow line. In the non-actuated state of the actuating element, the fluid pump delivers new fluid into the fluid reservoir through the inlet valve, whereas no fluid can pass from the fluid reservoir through the outlet valve to the patient port. This is then possible only when the actuating element is actuated, as a result of which the inlet valve blocks the inflow line and the outlet valve frees the outflow line, such that the pressurized fluid can flow from the fluid reservoir into the outflow line and to the patient port. As long as the fluid quantity in the fluid reservoir does not yet correspond to the bolus quantity, the actuating element is thus blocked, and no fluid can pass to the patient port. As soon as the bolus quantity is reached in the fluid reservoir, the actuating element is automatically freed, wherein an actuation of the actuating element has the effect that the inflow line is blocked and the outflow line is freed, as a result of which the pressurized fluid from the fluid reservoir shoots immediately into the less pressurized outflow line and flows to the patient port. The flow from the pump through the bolus fluid line is excluded during the bolus administration.

A bolus flow throttle is advantageously provided in the inflow line and suitably limits the flow rate in such a way that, before the fluid reservoir is completely filled again with the bolus quantity, a period of time has elapsed that prevents the patient from receiving an overdose upon renewed triggering of the bolus. In other words, this means that the cross section of the inflow line is reduced by the throttle in such a way that in the time after administration of a bolus, during which time a renewed administration could lead to an overdose, the quantity of fluid in the fluid reservoir is not sufficient to trigger a bolus. The fluid reservoir is again filled with the bolus quantity only when renewed triggering cannot lead to an overdose.

The trigger device is advantageously contained in a bolus fluid path that extends between a fluid delivery pump and the patient port. Parallel to the bolus fluid path, a main fluid path is provided for a continuous delivery of fluid to the patient port. The main fluid path can be provided with a main flow throttle for adjusting the flow rate. A filter is preferably arranged upstream of both flow paths or of each individual flow path. Alternatively or in addition, a filter is arranged downstream of both flow paths or of each individual flow path.

### BRIEF DESCRIPTION OF THE DRAWING FIGURES

An illustrative embodiment is explained in more detail below with reference to the figures, in which:

FIG. 1 shows a perspective view,

FIG. 2 shows an exploded view,

FIG. 3 shows a cross section in a first actuation state,

FIG. 4 shows a further cross section in the first actuation state,

FIG. 5 shows a further cross section in the first actuation state,

FIG. 6 shows a cross section in a second actuation state,

FIG. 7 shows a further cross section in the second actuation state,

FIG. 8 shows a further cross section in the second actuation state,

FIG. 9 shows a further cross section in the second actuation state,

FIG. 10 shows a cross section in a third actuation state,

FIG. 11 shows a perspective view in the third actuation state,

FIG. 12 shows a perspective view in a fourth actuation state,

FIG. 13 shows a further perspective view in the fourth actuation state,

FIG. 14 shows a perspective view in a fifth actuation state,

FIG. 15 shows a further perspective view in the fifth actuation state, and

FIG. 16 shows a schematic representation of the PCA device.

### DETAILED DESCRIPTION

The movable element 14 is a piston. The elastic element 16 is a piston spring. The actuating element 12 is an actuating button, wherein the two actuation blocking means 24, 28 are first and second button locks.

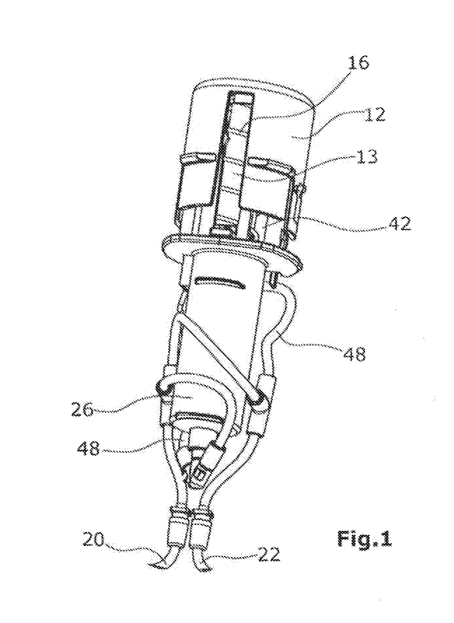

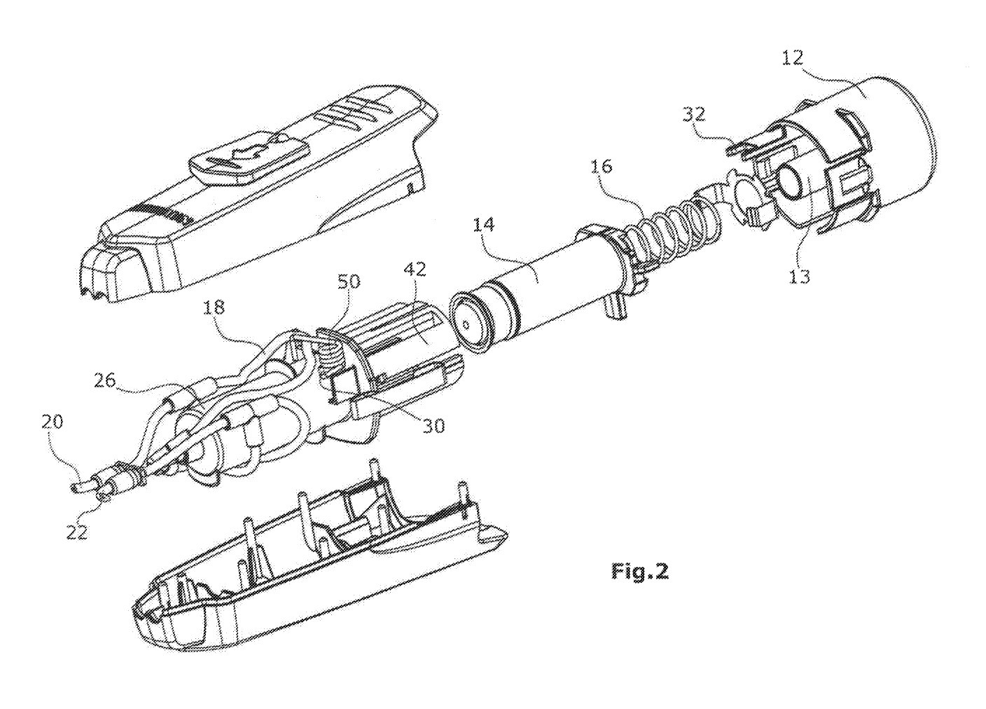

The actuating button 12 and the piston 14, which are pushed apart by a piston spring 16, can be seen in FIGS. 1 and 2. In the piston spring 16 is a helical spring which bears on the rear end of the piston 14 and surrounds a cylindrical inner neck 13 of the piston.

An actuating fork 32 protrudes forward in the distal direction from the actuating button 12. The actuating fork 32 is an integral part of the button 12. The actuating fork 32 is provided with a recess which engages with a spring arm 30 of the clamping spring 50.

The clamping spring 50 forms a closing mechanism for a segment of a hose 18 which can be connected to a fluid source via an inflow line 20 and to a patient via an outflow line 22.

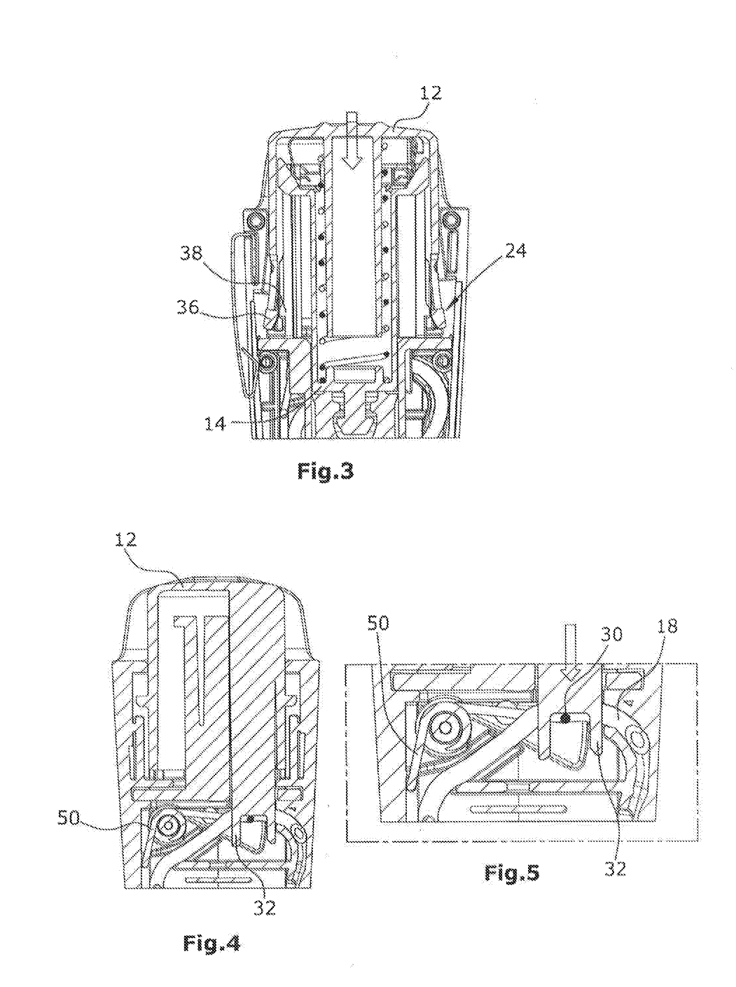

A piston lock 15 holds the piston in the upper, proximal position according to FIG. 3 (first actuation state). The piston lock 15 is formed as a latch mechanism by a latching lug 52 on a spring clip 54. The spring clip 54 is a part rigidly connected to the housing 42 of the actuating device. The latching lug 52 engages with a latching web 56 which protrudes laterally from the piston, transversely with respect to the longitudinal axis of the latter.

When the actuating button 12 is actuated by pressing the button 12 inward in the proximal direction (arrow direction in FIG. 3), the actuating fork 3 actuates the clamping spring 50 counter to the spring force of the latter. The spring arm 30 is thereby displaced, and the hose segment 18 is freed for fluid to flow through. FIGS. 4 and 5 show the actuated clamping spring 50 and the opened hose segment 18. Fluid from a fluid source connected to the inflow line 20 can flow through the hose segment 18 and the outflow line 22 in the direction of the patient.

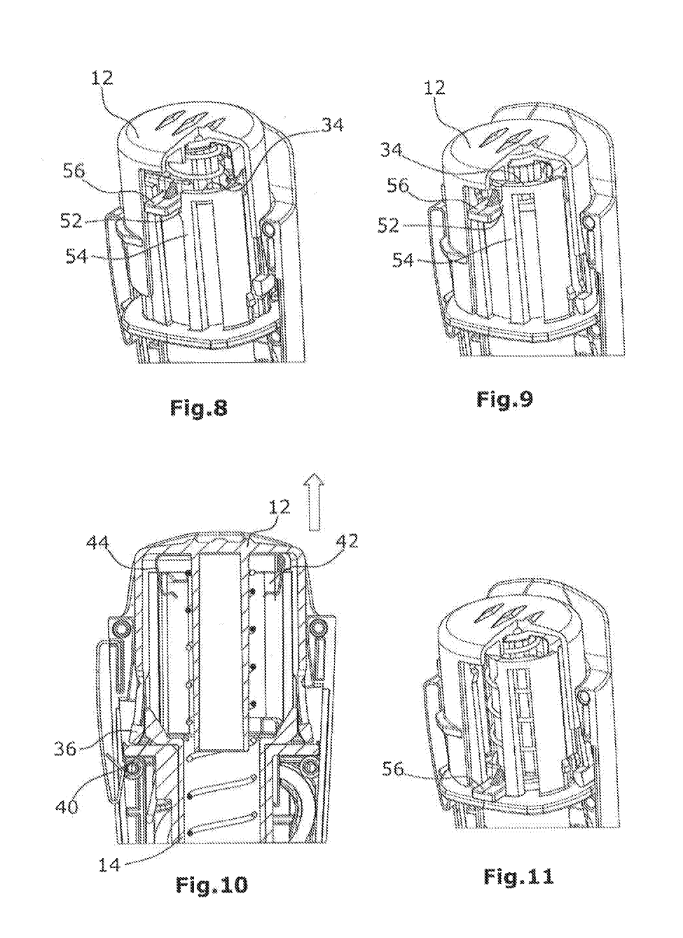

At the same time, the two engagement surfaces 34 of the actuating button 12 and of the piston 14 are pressed against each other, such that the pushing force of the button 12 is transmitted to the piston 14, and the piston 14 is thrust forward in the distal direction. The latching web 56 is advanced over the latching lug 52 of the spring clip 54 as is shown in FIGS. 8 and 9. As soon as the latching web 5 has overcome the latching lug 52 in the distal direction, the force of the piston spring, 16 exerts its effect on the piston 14 and pushes the latter forward in the distal direction. The button 12 is held in the pressed-in state by the first button lock 24.

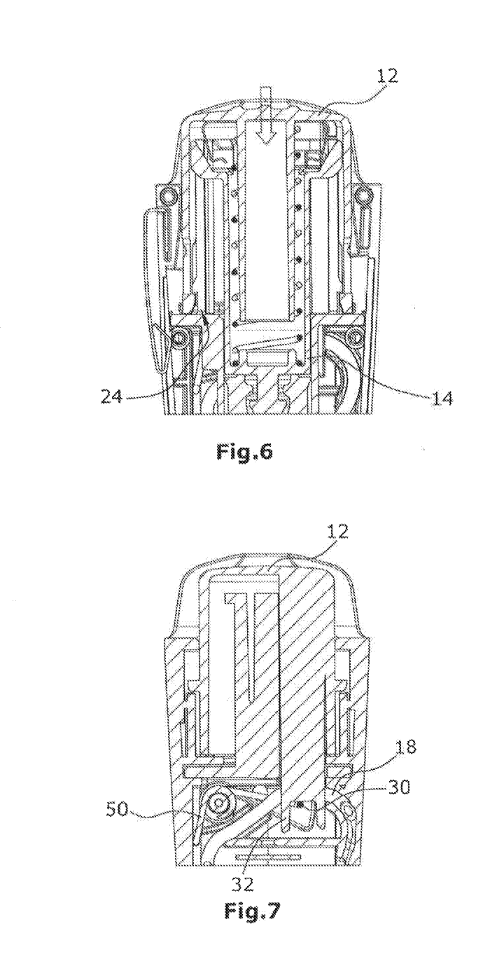

The first button lock 24 is formed by a latching pawl 36 on the button, which latching pawl 36 engages on a fixed part 38 of the housing 42. The housing part 38 is a projection. FIG. 3 shows how, when the button 12 is pressed in, and shortly before it reaches its lower position, the resilient latching pawl 36 of the button 12 is pushed over the housing part 38 (projection). When the lower position of the button 12 is reached, the latching pawl 36 is latched on the housing part 38, as can be seen in FIG. 6 (second actuation state). The first button lock 24 forms the abutment for the deployment of the three of the piston spring 16 onto the piston 14.

The piston 14 is thereby pressed into the bolus reservoir 26. As the piston 14 is pushed forward into the bolus reservoir 26, the liquid contained in the bolus reservoir is forced out.

FIG. 7 shows that, as the liquid is being forced out from the bolus reservoir 26, the clamping spring 50 is also further actuated and the hose segment 18 is freed. The fluid forced out from the bolus reservoir 26 is delivered to the outflow line 22 via a bolus line 48 and to a patient connected to the outflow line 22.

When the lower position of the piston 14 is reached, a first engagement surface 40 on a proximal collar of the piston 14 actuates the locking pawl 36 radially outwardly, as is shown in FIG. 10 (third actuation state). The latching pawl 36 is thus released from the fixed housing part 38. The first button lock 24 is therefore released automatically by the fact that the piston 14 reaches the lower position. The fluid is then forced completely out of the bolus reservoir 26. FIG. 11 shows that the latching web 56 has then reached its lowest position. The latching web 56 can thus serve as a level indicator. Based on the relative position of the level indicator 56, it is possible to deduce the level of the bolus reservoir 26. The button 12 is pushed back to its upper (proximal) position by the piston spring 16 in the proximal direction of the arrow in FIG. 10.

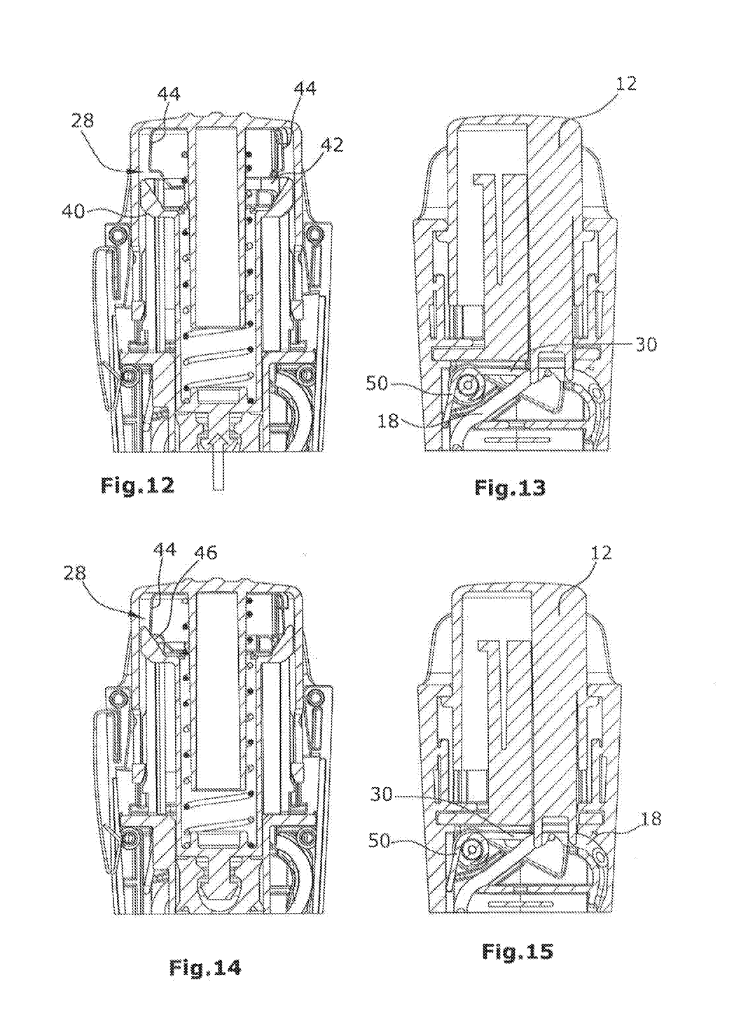

The button 12 is latched in its upper (proximal) position according to FIG. 12 by a second button lock 28 (fourth actuation state). The second button lock 28 is formed by a spring clip 44 which locks with respect to the housing 42 as is shown in FIG. 12. The spring clip 44 prevents the button 12 from being able to be pressed inward in the distal direction. An actuation of the actuating button 12 is thereby prevented.

While an actuation of the actuating button 12 is prevented by the second button lock 28, fluid flows from the fluid source (not shown in the figures) via the inflow line 20 and the bolus line 48 into the bolus reservoir 26. The fluid flowing into the bolus reservoir 26 presses the piston 14 upward in the proximal direction of the arrow in FIG. 12. The hose segment 18 is meanwhile closed by the clamping spring 50, since the button 12 is locked in the upper position by the second button lock 28. This is illustrated in FIG. 13. Therefore, no fluid can flow through the hose segment 18. The fluid pressure in the bolus reservoir 26 is therefore sufficiently increased in order to press the piston 14 upward and to fill the bolus reservoir 26.

As soon as the piston 14 reaches, the upper position as shown in FIG. 14, a second engagement surface 46 at the proximal collar of the piston 14 presses against the spring clip 44 and presses the latter radially inward, as a result of which the engagement between the spring clip 44 and the actuating button 12 is released (fifth actuation state). As soon as the bolus reservoir 26 is completely filled again with a bolus quantity of the fluid, the piston 14, in its upper position, frees the second button lock 28 via the second engagement surface 46. Thus, the button 12 is freed for a renewed actuation in order to trigger a further bolus. By contrast, actuation of the button 12 is prevented when the bolus reservoir 26 is not yet completely filled again.

FIG. 15 shows that in the completely filled state of the bolus reservoir 26, after the upper position of the piston 14 is reached, the hose segment 18 is still closed. It is only after a renewed actuation of the button 12 that the hose segment 18 is freed so that fluid can flow from the fluid source in the direction of the patient.

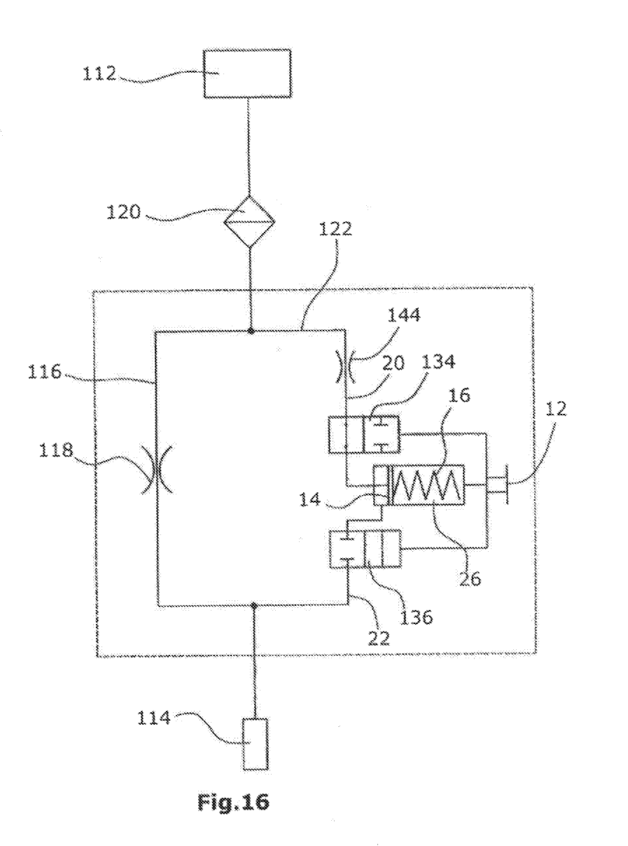

FIG. 16 shows the PCA device, which has a fluid delivery pump 112 and a patient port 114. A catheter inserted into a patient can be attached at the patient port 114. The fluid pump 112 delivers a painkiller through the fluid paths 116, 122 to the port 114. In this case, two fluid paths 116, 122 are provided between the pump 112 and the patient port 114. A first fluid path 116 forms a main fluid path, through which the fluid is delivered continuously from the pump 112 to the patient port 114. The flow cross section through the main fluid path 116 is defined by the main fluid throttle 118. A filter 120 is arranged between the main fluid throttle 118 and the fluid delivery pump 112.

Between the filter 120 and the patient port 114, a bolus fluid line 122 is connected in parallel to the main fluid line 116. The bolus fluid path 122 has an inflow line 20, connected to the filter 120 and to the pump 112, and an outflow line 22 connected to the patient port 114. Between the inflow line 20 and the outflow line 22, a fluid reservoir 26 is formed which is connected to the latter. The inflow line 20 and the outflow line 22 each open into the fluid reservoir 26.

A piston 14, movable counter to the force of a spring 16, is provided in the fluid reservoir 26. The fluid reservoir 26 is in this case formed as a cylinder in which the piston 14 is sealingly guided. Fluid flowing through the inflow line 20 into the fluid reservoir 26 displaces the piston 14 counter to the force of the spring 16 when the pressure applied by the fluid delivery pump 112 is sufficient to overcome the spring force.

Between the fluid reservoir 26 and the pump 112, the inflow line 20 has an inlet valve 134. Between the fluid reservoir 26 and the patient port 114, the outflow line 22 has an outlet valve 136. The inlet valve 134 and the outlet valve 136 each have a blocking position in which the respective fluid path 20, 22 is blocked, and a passage position in which the respective fluid path 20, 22 is freed. The inlet valve 134, the fluid reservoir 26 and the outlet valve 136 are connected to an actuating element 12, which at the same time actuates the inlet valve 134, the piston 14 and the outlet valve 136. The outlet valve 136 can be the closing mechanism for the hose segment 18.

The inlet valve 134 and the outlet valve 136 are displaced by the actuating element 12 transversely with respect to the direction of flow of the fluid. In the non-actuated state of the actuating element 12 as shown in FIG. 16, the inlet valve 134 frees the inflow line 20 and the outlet valve 136 blocks the outflow line 22. In this way, fluid delivered by the fluid delivery pump 112 can flow through the inlet valve 134 and through the inflow line 20 into the fluid reservoir 26, where it displaces the piston 14. By contrast, the fluid cannot flow from the fluid reservoir 26 to the patient port 114, since the outflow line 22 is blocked by the outlet valve 136.

1. An actuating device for administering a bolus, the actuating device comprising:
an actuating element, which is displaceable from an upper position to a lower position in order to actuate a closing mechanism which opens a hose segment in the lower position of the actuating element and closes the hose segment in the upper position of the actuating element, wherein the hose segment can be connected to a fluid source via an inflow line and to a patient via an outflow line,
a movable element, which is displaceable from an upper position to a lower position, independently of the actuating element, or is deformable,
a blocking element which locks the movable element in the upper position and which can be released by the actuating element by displacement thereof in a direction of the lower position,
an elastic element, which pushes the actuating element and the movable element apart,
a first actuation blocking element, which locks the actuating element in the lower position thereof and can be released by the movable element in the lower position thereof,
a bolus reservoir for holding a bolus quantity of a fluid, the bolus reservoir comprising a volume wherein the volume of the bolus reservoir can be reduced by displacement or deformation of the movable element in the direction of the lower position thereof, and
a second actuation blocking element, which locks the actuating element in the upper position thereof and can be released by the movable element in the upper position thereof.

2. The actuating device according to claim 1, wherein the bolus reservoir is connected to the inflow line and the outflow line via a bolus line.

3. The actuating device according to claim 2, wherein the hose segment has a lower flow resistance than the bolus line.

4. The actuating device according to claim 1, wherein the closing mechanism has a clamping spring which clamps off the hose segment, the clamping spring exerting a spring force that counteracts a displacement of the actuating element in the direction of the lower position thereof.

5. The actuating device according to claim 4, wherein the clamping spring has a spring arm that clamps off the hose.

6. The actuating device according to claim 5, wherein the actuating element has an actuating fork that actuates the spring arm.

7. The actuating device according to claim 1, wherein the blocking element has a latch mechanism with a latching lug which is arranged on a spring clip and which holds the movable element.

8. The actuating device according to claim 7, wherein the movable element has a latching web that engages on the latching lug.

9. The actuating device according to claim 1, wherein the movable element and the actuating element each have an engagement surface, wherein the engagement surfaces can be placed against each other in order to transmit a force from the actuating element to the movable element.

10. The actuating device according to claim 1, wherein the elastic element displaces the movable element after release of the blocking element by the actuating element.

11. The actuating device according to claim 1, wherein the first actuation blocking element has a resilient latching pawl which exerts a spring force, the latching pawl being arranged on the actuating element and which engages on a fixed housing part.

12. The actuating device according to claim 11, wherein the movable element has a first engagement surface which actuates the latching pawl counter to the spring force of the latching pawl.

13. The actuating device according to claim 1, wherein the second actuation blocking element has a spring clip which exerts a spring force and which locks the actuating element with respect to a housing of the actuating device.

14. The actuating device according to claim 13, wherein the movable element has a second engagement surface which actuates the spring clip counter to the spring force of the spring clip.

15. The actuating device according to claim 1, wherein at least a part of the movable element is guided with a sliding movement in the bolus reservoir.

16. The actuating device according to claim 1, wherein the movable element is a piston or a balloon.

17. The actuating device according to claim 1, wherein the movable element is mounted in the bolus reservoir in such a way as to be displaceable or deformable counter to a force of an elastic element.

18. The actuating device according to claim 1, wherein the actuating element can be actuated along a first actuation path and along a second actuation path that goes beyond the first actuation path, wherein the actuating element, when traveling along the first actuation path, permits a manual administration of a first bolus quantity, and it is only when traveling along the second actuation path that it triggers an automatic administration of a second bolus quantity.

19. The actuating device according to claim 1, wherein an inlet valve is provided in the inflow line and an outlet valve is provided in the outflow line, wherein the inlet valve and the outlet valve are connected to the actuating element in such a way that, in a non-actuated state of the actuating element, the inlet valve frees the inflow line and the outlet valve blocks the outflow line, and in an actuated state of the actuating element, the inlet valve blocks the inflow line and the outlet valve frees the outflow line.

20. The actuating device according to claim 1, wherein the inflow line is provided with a bolus flow throttle.

21. A patient-controlled analgesia device (PCA device) with a fluid delivery pump, a patient port, a main fluid path formed between the fluid delivery pump and the patient port, and a bolus fluid path which is arranged between the fluid delivery pump and the patient port and is parallel to the main fluid path, wherein an actuating device according to claim 1 is provided in the bolus fluid path.

22. The PCA device according to claim 21, wherein the main fluid path has a main flow throttle.

23. The PCA device according to claim 21, wherein the main fluid path and/or the bolus fluid path have/has a manually adjustable filling throttle.
